(19) United States
(12) Patent Application Publication
(10) Pub. No.: US 2018/0064613 A1
(43) Pub. Date: Mar. 8, 2018

(54) SEQUENTIAL MATERIAL DEPOSITION FOR DESENSITIZATION AND REMINERALIZATION OF TEETH

(71) Applicant: South Dakota Board of Regents, Pierre, SD (US)

(72) Inventors: Grigoriy Sereda, Vermillion, SD (US); Joshua Brower, Sioux Falls, SD (US)

(21) Appl. No.: 15/707,742

(22) Filed: Sep. 18, 2017

Related U.S. Application Data
(63) Continuation of application No. 15/189,228, filed on Jun. 22, 2016, now abandoned.
(60) Provisional application No. 62/183,220, filed on Jun. 23, 2015.

(51) Int. Cl. A61K 6/00; A61Q 11/00; A61K 8/34; A23G 4/06; A61C 19/06; A61C 19/08; A61K 8/73; A61K 8/60; A61K 8/365; A61K 8/29; A61K 8/26; A61K 8/25; A61K 8/24; A61K 8/11; A61K 8/02

(52) U.S. Cl. CPC A61K 6/0047; A61K 2800/92; A61K 8/347; A23G 4/06; A23G 4/064; A61C 19/063; A61C 19/08; A61K 6/0008; A61K 8/736; A61K 8/735; A61K 8/733; A61K 8/604; A61K 8/365; A61C 5/62; A61K 8/29; A61K 8/26; A61K 8/25; A61K 8/24; A61K 8/11; A61K 8/0241; A61K 6/007; A61K 2800/28; A61K 2800/412; A61K 2800/413; A61K 2800/612; A61K 2800/884; A61Q 11/00

(57) ABSTRACT

The present invention relates to a method of remineralizing and desensitizing teeth utilizing the sequential steps of first applying to the teeth a first component that attaches to the teeth and secondly applying to the teeth treated with the first component a second component that attaches to the first component.

### CROSS REFERENCE TO RELATED APPLICATIONS

This application claims priority from U.S. Provisional Application Ser. No. 62/183,220, filed Jun. 23, 2015 as well as U.S. Utility application Ser. No. 15/189,228, filed Jun. 22, 2016.

### FIELD OF THE INVENTION

The present invention relates generally to dental health and, more specifically, to a method of applying to exposed dentinal tubules two or more agents that act in a synergistic fashion to occlude the open tubules and decrease dental sensitivity and to serve as a scaffold for remineralization.

### BACKGROUND OF THE INVENTION

Demineralization of the teeth leads to hypersensitivity and dental caries, both significant public health concerns. Demineralization is primarily caused by acids present in the mouth, whether through the intake of acidic foods or beverages, the production of acids by bacteria present in the mouth, or the regurgitation of stomach acids into the mouth. Demineralization exposes the dentinal tubules allowing access to dental nerves. It also weakens the hard surfaces of the teeth, increasing the incidence of dental caries.

There are currently three materials on the market that are proved to facilitate tooth remineralization by supplying hydroxyapatite to the tooth surface. The first material is Casein PhosphoProtein Amorphous Calcium Phosphate (CPP-ACP, Recaldent®), which is the subject of an issued US patent by an Australian professor Eric Reynolds (1). The active component is a synthetic complex of amorphous hydroxyapatite with a phosphoprotein that binds both the amorphous mineral and the tooth surface with the serine phosphate residues in the protein's sequence. This complex is naturally present in mammals' milk and has shown to promote tooth remineralization as a component of Ml Paste (2), chewing gum (3), mouth rinse (3), and sealants (3), and when added to bovine milk (3,4). In addition, CPP-ACP decreases tooth hypersensitivity by occluding the dentinal tubules and protecting the tooth's pulp chamber from penetration by saliva and food components (5).

The second material NovaMin® was patented in 2010 (6) as the remineralizing (7) and desensitizing (8) component of Dr. Collin's Restore® toothpaste. This material is described as sodium calcium phosphosilicate of unspecified chemical structure and composition (6). Despite the lack of a proper description of NovaMin®, one can guess that it comprises some kind of inorganic particles containing ionic components of hydroxyapatite that somehow bind to the tooth surface.

The natural ability of hydroxyapatite nanoparticles to blend with the tooth surface was utilized in the third material, Biorepair®, and similar desensitizing and remineralizing toothpastes (9). Currently the leading desensitizing product available commercially delivers its desensitizing effect by the hydrolysis of a relatively toxic active component, tin difluoride.

All existing dentifrices are based on the simultaneous application of active components to the tooth surface. The active components are often either non-natural materials (silica-based), or require the presence of parabene preservatives, unnecessarily exposing the consumer to potentially harmful chemicals.

The present invention makes use of two components that act in a synergistic fashion, utilizing their chemical or physical interactions to result in crosslinking, specific layer-by-layer deposition of components, or a combination of both cross-linking and layer-by-layer deposition. The components lead to occlusion of the tubules both relieving sensitivity and serving as a scaffold for remineralization. Comparing FIG. 1 to FIG. 2 demonstrates the difference between using just a leading toothpaste (FIG. 1) and brushing with a leading toothpaste after pre-treatment with chondroitin (FIG. 2). The method presently disclosed is able to withstand thirty seconds of ultrasonic challenge, an important characteristic considering the proliferation of sonication-based toothbrushes. The components can be supplied in foods, dentures, retainers, or mouth guards rather than the classic dentifrices and mouthwashes commonly used for maintenance of dental hygiene. The components can be supplied by either different dentifrices (such as mouthwash and toothpaste), or different domains of the same dentifrice (such as different layers of a chewing gum, or microcapsules embedded into a chewing gum), or by saliva. The energy of chewing can be harnessed to mix the components and initiate their layer-by-layer deposition, cross-linking, or both.

### BRIEF DESCRIPTION OF THE DRAWINGS

FIG. 1 shows no adhesion of a leading titanium dioxide-containing toothpaste to dentin.

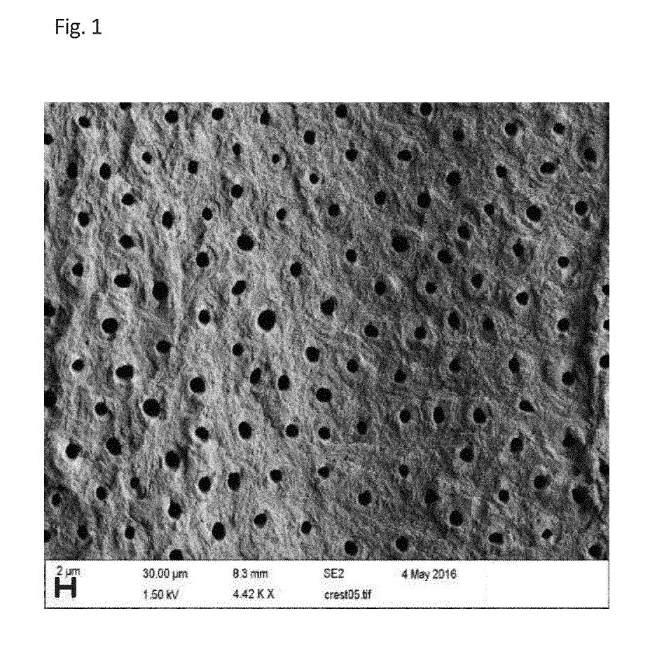

FIG. 2 shows adhesion of a leading titanium dioxide-containing toothpaste to dentin pre-treated with chondroitin. The adhesion achieved by this method is resistant toward challenge with 30 seconds of ultrasound.

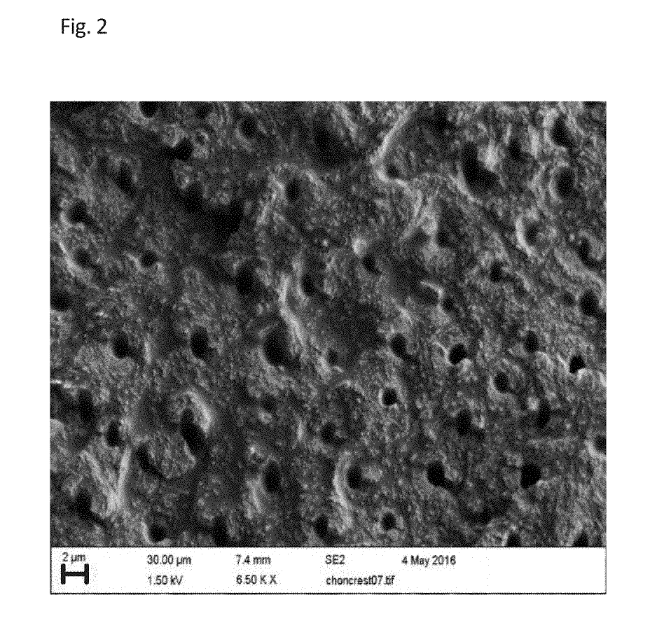

FIG. 3 shows occlusion of dentinal tubules by carboxylated silica nanoparticles. The adhesion achieved by this method is resistant toward challenge with 30 seconds of ultrasound.

FIG. 4 shows occlusion of dentinal tubules by the sequential deposition of chitosan and alginate. The adhesion achieved by this method is resistant toward challenge with 30 seconds of ultrasound.

FIG. 5 shows the dentin surface following dentifrice application with or without chondroitin sulfate pre-treatment. (a) The surface after treatment with toothpaste and 30 seconds of ultrasonication. (b) Sensodyne® treatment followed by a 30 seconds of ultrasonication. (c) 1 min chondroitin sulfate pretreatment followed by application of a leading titanium dioxide-containing toothpaste and a 30 seconds of ultrasonication. (d) 1 min chondroitin sulfate pretreatment followed by application of Sensodyne® ProNamel® and a 30 second ultrasonication.

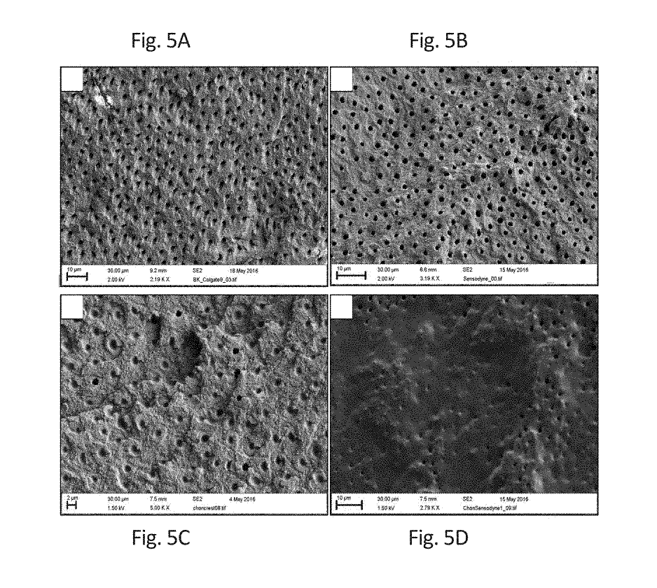

FIG. 6 shows little occlusion of dentinal tubules by 1% arginate.

FIG. 7 shows significant occlusion of dentinal tubules by 1% arginate followed by treatment with calcium ions. The adhesion achieved by this method is resistant toward challenge with 30 seconds of ultrasound.

FIG. 8 shows tubule occlusion by sequential treatment with 1% chitosan and 10% sodium citrate. The adhesion achieved by this method is resistant toward challenge with 30 seconds of ultrasound.

FIG. 8 shows no adhesion of bentonite to dentin.

FIG. 9 shows adhesion of bentonite to dentin pretreated with calcium ions. The adhesion achieved by this method is resistant toward challenge with 30 seconds of ultrasound.

FIG. 10 shows adhesion of bentonite to dentin pretreated with calcium ions at a higher magnification level than FIG. 9.

### SUMMARY OF THE INVENTION

The subject of the invention is delivery of remineralizing, desensitizing, and other functional components to the tooth surface by the sequential deposition of oppositely charged (or non-electrostatically interacting) components on the tooth surface, especially inside dentinal tubules. Those components include nanoparticles, microparticles, polymer films, phospholipids, and small molecules, pristine or modified by remineralizing, desensitizing, antibacterial, fluoride-supplying, and other functional groups. The components are contained in different dentifrices (toothpaste, mouth rinse, chewing gum, dental floss, toothpicks, etc.), dentures, retainers, mouth guards, or foods and are deposited on the tooth surface by sequential application of the listed dentifrices, consumption of foods, or combinations thereof. The positively charged components may include chitosan, polylysine, arginine, calcium cations, proteins, functionalized nano- and micro-particles, and polymer films. The negatively charged components may include bare and functionalized silica particles, chondroitin, hyaluronic acid, alginate, nucleic acids, citrates, and polymer films. The neutral components may include phospholipids, particles of calcium citrate, and particles of hydroxyapatite. The components form the functional material at the tooth surface and inside the dentinal tubules by the mechanism of layer-by-layer deposition, cross-linking, or combinations of both. In certain instances, the energy of chewing can be harnessed to mix the components and initiate their layer-by-layer deposition, cross-linking, or both.

The new methods enable the synergistic effect of desensitizing and remineralizing due to the known effect of guided remineralization of hydroxyapatite on matrixes of biopolymers. Thus, deposition of hydroxyapatite on chitosan-chondroitin matrixes is one of the chief foci of bone tissue bioengineering (10).

It is an object of the present invention to prevent or ameliorate the incidence of dental caries and treat hypersensitivity of the teeth, thereby improving the quality of life of subjects and reducing health care costs. Another object of the present invention is to provide products that can be used not only by health care providers when treating patients but also by subjects on their own. A further object of the invention is to open up an entirely new market of tooth-healing foods.

### DETAILED DESCRIPTION OF PREFERRED EMBODIMENTS

The approach of the present invention of sequential application utilizes advantages of the layer-by-layer deposition of materials chemically or physically bound to one another. This methodology will create a robust desensitizing seal inside the dentin tubules. Since the deposited materials can be imparted with additional desensitizing, antibacterial, remineralizing, and drug-delivering (such as fluoride) properties, the proposed technique is a significant improvement versus existing dentifrice materials. In addition, the capability of multilayer films to deliver functional materials (fluoride, calcium, potassium, eugenol, etc.) to the tooth surface and saliva in a time-controlled fashion is especially critical for the prolonged periods between meals when the natural saliva (stationary saliva) flow is the lowest, least alkaline, and minimally protective to the tooth. The employed components can be selected from a wide range of non-toxic natural materials such as chondroitin, calcium citrate, chitosan, and strawberry DNA. A significant innovation of the new method is utilizing the synergistic effect of desensitization and remineralization due to the guided deposition of hydroxyapatite on the scaffolds formed by the desensitizing agents in the process of dentinal tubule occlusion. Utilization of a food-grade alginate cross-linked by calcium ions, may allow for the use of culinary recipes instead of medicated compositions. The term “dentifrice” as used throughout this description, denotes a paste, gel, or liquid formulation. The dentifrice may be in any desired form, such as toothpaste (including, but not limited to, deep striped, surface striped, multi-layered, having a gel surround the paste); powder, beads; mouthwash; mouth rinses; lozenge; dental gel; periodontal gel; liquid suitable for painting a dental surface; chewing gum; dissolvable, partially dissolvable, or non-dissolvable film or strip; wafer; wipe or towelette; implant; foam; troche; dental floss; liquid formulated for oral application in a small, portable nebulizer (spray bottle); liquid formulated for oral application in a small, portable, drop-generating bottle; soft pliable tablet (“chew”); or any combinations thereof.

The invention will now be described with reference to the following non-limiting examples.

### Example 1

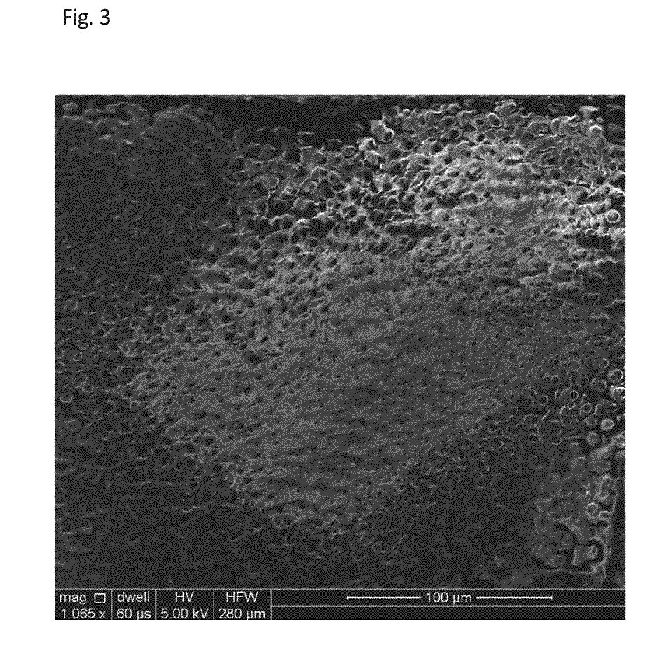

Application 1: Toothpaste containing potassium carboxylate-functionalized silica nanoparticles that, due to the presence of carboxy-groups, bind to the tooth surface occluding the tubules and additionally delivering the desensitizing material (potassium cation) to the tooth. See FIG. 3.

Application 2: Mouth rinse containing antibacterial chitosan nanoparticles (positively charged) that cover the already deposited silica nanoparticles (negatively charged) and seal the tubules enhancing the desensitizing effect.

### Example 2

Application 1: Mouth rinse containing antibacterial chitosan (positively charged) that electrostatically interacts with dentin.

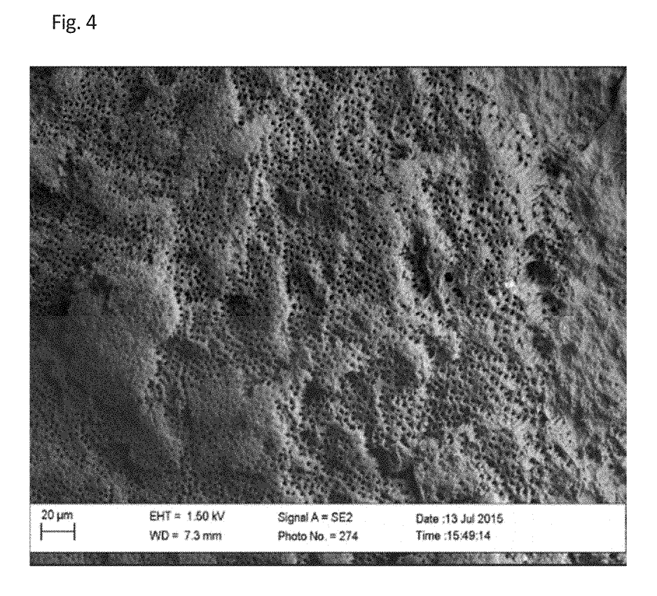

Application 2: Mouth rinse containing alginate (negatively charged) that covers the already deposited chitosan (positively charged) and seals the tubules enhancing the desensitizing effect. Any concentration of chitosan and alginate up to about 20% can be used. The deposited chitosan provides a site for remineralization due to its affinity for calcium ions. See FIG. 4.

### Example 3

Application 1: Toothpaste containing nanocrystals of hydroxyapatite coated with chondroitin sulfate that bind to the tooth surface due to the presence of carboxy-groups in chondroitin and deliver the remineralizing material (hydroxyapatite) to the tooth.

Application 2: Mouth rinse containing antibacterial chitosan nanoparticles (positively charged) that cover the already deposited chondroitin (negatively charged) and seal the tubules enhancing the desensitizing effect. In addition, the chitosan-chondroitin scaffold will promote remineralization by hydroxyapatite and its components from saliva.

Application 3: Dental floss or toothpicks functionalized by hydroxyapatite or calcium citrate will provide additional material for remineralization.

### Example 4

Application 1: Toothpaste containing nanocrystals of hydroxyapatite coated with DNA, including without limitation strawberry DNA, that bind to the tooth surface due to the presence of phosphate-groups in the DNA and deliver the remineralizing material (hydroxyapatite) to the tooth.

Application 2: Dental floss modified with eugenol-loaded antibacterial chitosan nanoparticles (positively charged) that cover the already deposited DNA (negatively charged) and seal the tubules enhancing the desensitizing effect, which is additionally enforced by eugenol.

### Example 5

Application 1: Toothpaste containing nanocrystals of calcium citrate grown in the presence of chondroitin sulfate that bind to the tooth surface due to the presence of carboxy-groups in chondroitin and deliver the remineralizing material (calcium) to the tooth.

Application 2: Mouth rinse containing antibacterial chitosan nanoparticles (positively charged) that cover the already deposited chondroitin (negatively charged) and seal the tubules enhancing the desensitizing effect. In addition, the chitosan-chondroitin scaffold will promote remineralization by hydroxyapatite and its components from saliva.

Application 3: Dental floss or toothpicks functionalized by hydroxyapatite or calcium citrate will provide additional material for remineralization.

### Example 6

Application 1: Mouth rinse containing potassium chondroitin sulfate that binds to the tooth surface due to the presence of carboxy-groups in chondroitin, and delivers the desensitizing material (potassium) to the tooth.

Application 2: Mouth rinse containing antibacterial chitosan fluoride nanoparticles (positively charged) that cover the already deposited chondroitin (negatively charged), seal the tubules enhancing the desensitizing effect, and deliver fluoride strengthening the tooth. In addition, the chitosan-chondroitin scaffold will promote remineralization by hydroxyapatite and its components from saliva.

Application 3: Dental floss or toothpicks functionalized by hydroxyapatite or calcium citrate will provide additional material for remineralization.

### Example 7

Application 1: Mouth rinse containing potassium chondroitin sulfate that binds to the tooth surface due to the presence of carboxy-groups in chondroitin, and delivers the desensitizing material (potassium) to the tooth. Dentin samples were submersed for 15 seconds or more in the prepared chondroitin sulfate solution at −1° C. to 10° C. and rinsed with water.

Application 2: An existing toothpaste containing titanium dioxide particles that bind to the chondroitin layer due to the complexation of carboxy-groups with titanium, seal the tubules, and thus deliver the desensitizing effect to the tooth. The chondroitin sulfate-treated samples were gently brushed with either a leading titanium dioxide-containing toothpaste, or Sensodyne® for 5 seconds or more and then rinsed with water.

The adhesion achieved by this method is resistant toward 30 seconds of ultrasound challenge. We have found that the occlusion induced by Sensodyne® alone does not withstand the ultrasound challenge.

### Example 8

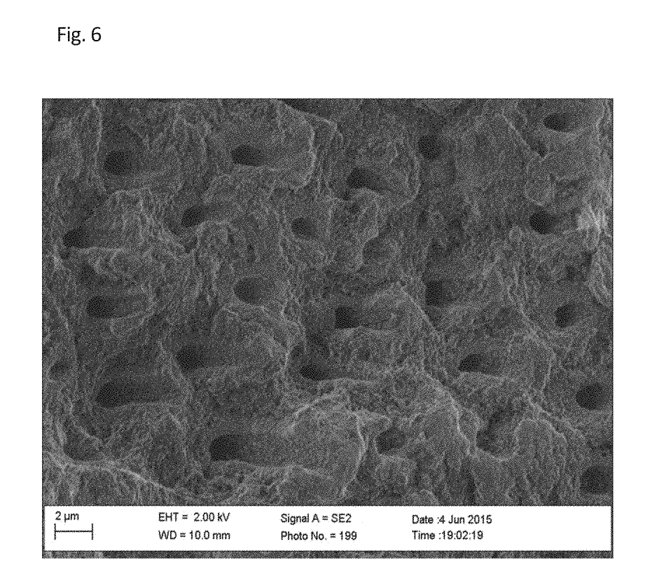

Application 1: Mouth rinse containing potassium alginate that binds to the tooth surface due to the presence of carboxy-groups in alginate. Dentin samples were treated with an aqueous alginate solution (0.001-10% alginate) mimicking a mouth rinse for at least one second. See FIG. 6.

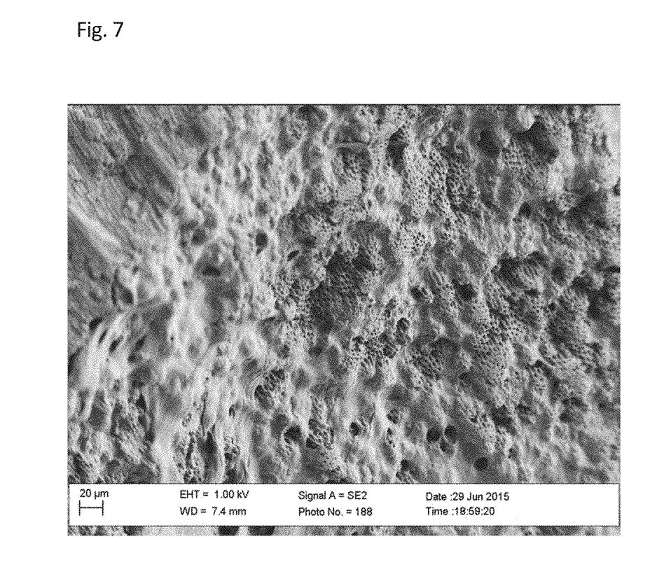

Application 2: Mouth rinse containing calcium ions that cross-link the alginate polymer due to its affinity for calcium-ions and delivers desensitizing effects to the tooth. Dentin samples pre-treated with alginate (Example 1) were treated with an aqueous calcium salt (0.001-40 w % of Ca²⁺) mimicking a mouth rinse for at least 1 second. See FIG. 7.

### Example 9

Application 1: Mouth rinse containing a chitosan-phosphate gel that penetrates dentinal tubules via capillary action.

Application 2: Temperature rises to the body temperature, which hardens the gel and causes tubule occlusion.

### Example 10

Application 1: Consumption of a food containing potassium alginate that binds to the tooth surface due to the presence of carboxy-groups in alginate and delivers the desensitizing material (potassium) to the tooth.

Application 2: Consumption of a calcium-rich food such as milk. Calcium cations cross-link the alginate inside the dentinal tubules, forming an occlusion matrix loaded with a remineralizing component.

### Example 11

Application 1: Wearing dentures, athletic mouth guards, or overnight retainers or mouth guards charged with alginate nanoparticles that bind to the tooth surface due to the presence of carboxy-groups in alginate, and deliver the desensitizing material (potassium) to the tooth.

Application 2: Consumption of a calcium-rich food such as milk. Calcium cations cross-link the alginate inside the dentin tubules, forming an occlusion matrix loaded with a remineralizing component.

### Example 12

Application 1: Mouth rinse containing antibacterial chitosan (positively charged) that electrostatically interacts with dentin.

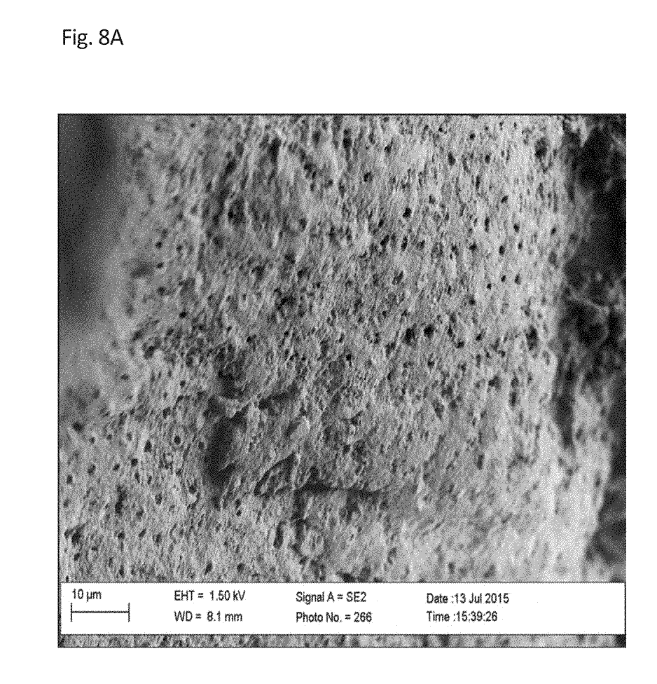

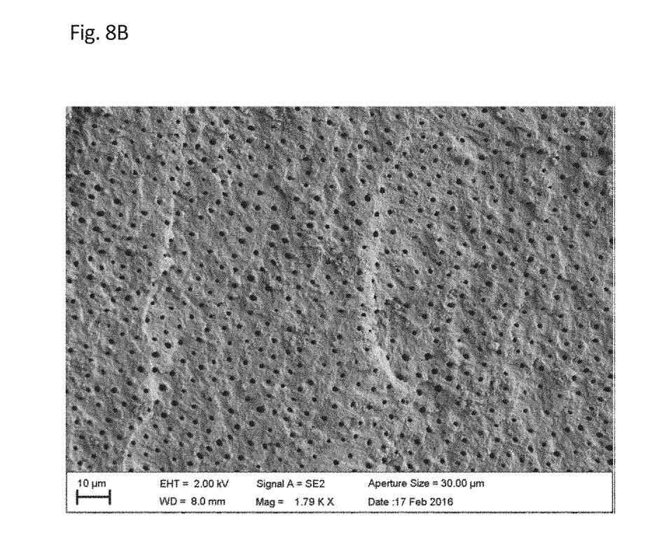

Application 2: Mouth rinse containing citrate-ions that cross-link the already deposited chitosan and seal the tubules delivering the desensitizing effect. Additionally, presence of citrate ion strengthens the bone and provides a site for remineralization due to its affinity to calcium ions. See FIG. 8.

### Example 13

Application 1: Chewing gum containing layers of potassium alginate that bind to the tooth surface due to the presence of carboxy-groups in alginate and deliver the desensitizing material (potassium) to the tooth.

Application 2: Additional layers of the same chewing gum containing calcium chondroitin that is mixed with the alginate by the energy of chewing and cross-linked. The tubules are occluded, and the desensitizing material (potassium) is delivered to the tooth.

### Example 14

Application 1: Chewing gum containing microcapsules of potassium alginate that bind to the tooth surface due to the presence of carboxy-groups in alginate and deliver the desensitizing material (potassium) to the tooth.

Application 2: Additional layers of the same chewing gum containing microcapsules of calcium chondroitinate that is mixed with the alginate by the energy of chewing and cross-linked. The tubules are occluded, and the desensitizing material (potassium) is delivered to the tooth.

### Example 15

Application 1: Mouth rinse containing calcium ions that are adsorbed on the tooth surface. Dentin samples were treated with an aqueous calcium salt (0.001-40 w % of Ca²⁺) mimicking a mouth rinse for at least 1 second.

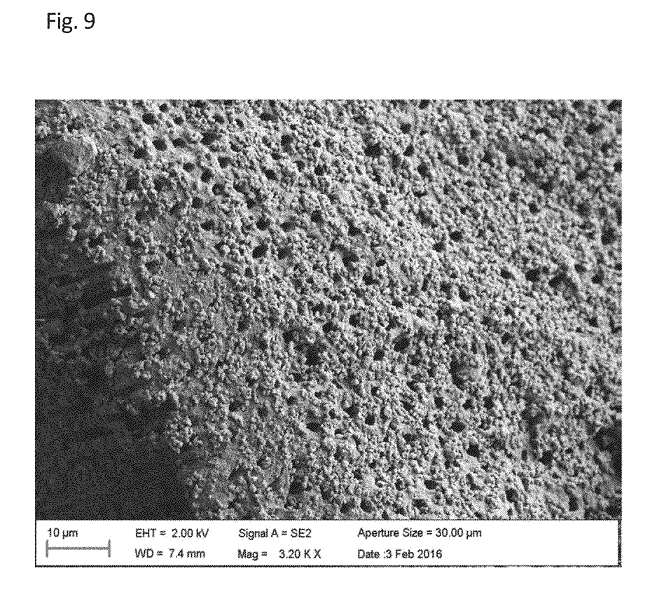

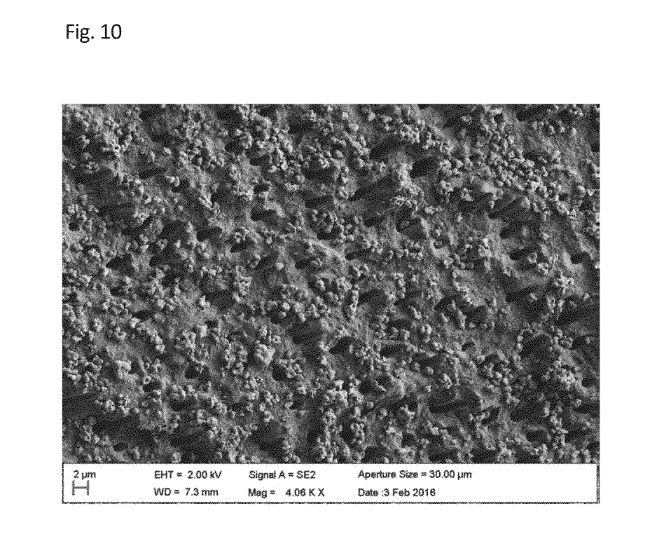

Application 2: A toothpaste containing particles of bentonite that bind to the deposited calcium due to the complexation of negatively charged groups of bentonite with calcium, occlude the tubules, and thus deliver the desensitizing effect to the tooth. The Ca²⁺-pretreated dentin samples (Application 1) were brushed with bentonite to model a bentonite-based toothpaste for at least 2 seconds. See FIGS. 9 and 10.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

1. Reynolds. E. Stabilized Calcium Phosphate Complexes. U.S. Pat. No. 8,609,071 B1.
2. Zhang, Q.; Zou. J.; Yang. R.; Zhou, X. Remineralization effects of casein phosphopeptideamorphous calcium phosphate cre'me on artificial early enamel lesions of primary teeth International Journal of Paediatric Dentistry 2011, 21, 374-381.
3. Gurunathan, D.; Somasundaram, S.; Kumar, S. A. Casein phosphopeptideamorphous calcium phosphate: a remineralizing agent of enamel. Australian Dental Journal 2012, 57, 404-408.
4. Walker. G. D.; Cai. F.; Shen, P.; Bailey, D. L.; Yuan, Y.; Cochrane, N. J.; Reynolds, C.; Reynolds. E. C. Consumption of milk with added casein phosphopeptideamorphous calcium phosphate remineralizes enamel subsurface lesions in situ Australian Dental Journal 2009, 54, 245-249.
5. Reynolds, E. Treatment for Sensitive Teeth international Patent, 1994, WO 94/00146.
6. Zaidel. L.; Prencipe, M.; Chopra, S. K. Oral Compositions for Treating Tooth Sensitivity and Methods of Use and Manufacture Thereof International Patent, 2010. WO 2010/115041 A2.
7. Diamanti, i.; Koletsi-Kounari, H.; Mamai-Homata, E.; Vougioukiakis, G. In vitro evaluation of fluoride and calcium sodium phosphosilicate toothpastes, on root dentine caries lesions J. Dentistry 2011, 39, 619-628.
8. Narongdej, T.; Sakoolnamarka, R.; Boonroung, Th. The Effectiveness of a Calcium Sodium Phosphosilicate Desensitizer in Reducing Cervical Dentin Hypersensitivity: A Pilot Study JADA, 2010, 141, 8, 995-999.
9. Tschoppe. P.; Zandim. D. L.; Martus. P.; Kielbassa. A. M. Enamel and dentine remineralization by nano-hydroxyapatite toothpastes J. Dentistry 2011, 39.430-437.
10. Rhee S. H., Tanaka, J. Self-assembly phenomenon of hydroxyapatite nanocrystals on chondroitin sulfate. Journal of Materials Science: Materials in Medicine 2002, 13, 597-600.

What is claimed is:

1. A method of desensitizing teeth, comprising the steps of:
(a) applying to the teeth a first component selected from the group consisting of
(b) nanoparticles, microparticles, polymer films, phospholipids and small molecules that attaches to the teeth; and
(c) applying to the teeth treated with the first component a second component that cross-links to the first component.

2. The method of claim 1, wherein said first component comprises negatively charged components and said second component comprises positively charged components.

3. The method of claim 2, wherein said first component is selected from the group consisting of bare and functionalized silica particles, chondroitin, hyaluronic acid, nucleic acids, citrates, and polymer films and wherein said second component is selected from the group consisting of chitosan, polylysine, arginine, calcium cations, proteins, functionalized nano- and micro-particles, and polymer films.

4. The method of claim 1, wherein said first component comprises positively charged components and said second component comprises negatively charged components.

5. The method of claim 4, wherein said first component is selected from the group consisting of chitosan, polylysine, arginine, calcium cations, proteins, functionalized nano- and microparticles, and polymer films and wherein said second component is selected from the group consisting of bare and functionalized silica particles, chondroitin, hyaluronic acid, nucleic acids, citrates, and polymer films.

6. The method of claim 1, wherein either said first or second component further comprises nanoparticles, microparticles, polymer films, phospholipids, and small molecules, pristine or modified by remineralizing, desensitizing, antibacterial, fluoride-supplying, and other functional groups.

7. The method of claim 1, wherein said first and second components are contained in different dentifrices, such as toothpaste, mouth rinse, chewing gum, dental floss, toothpicks, dentures, retainers, and the like, or foods, and are deposited on the tooth surface by sequential application of the listed dentifrices or consumption of foods, or combinations of thereof.

8. The method of claim 1, wherein said cross-linked first and second components form an occlusive deposit sufficiently persistent and durable to withstand an ultrasound challenge.
